SEQUENCE LISTING (43 sequences)

SEQ ID NO: 1 - DNA, 37 bp, Artificial Sequence
  MH-gap-F1 primer
tgctccagctaaaagcgaaggnnnnnaaacagttgtt

SEQ ID NO: 2 - DNA, 40 bp, Artificial Sequence
  MH-gap-F2 primer
actgtttagctcctattgccaacgnnnnngaaaaaaactt

SEQ ID NO: 3 - DNA, 38 bp, Artificial Sequence
  MH-gap-R2 primer
gcctgcttttgcaccaataatannnnntgatacaattg

SEQ ID NO: 4 - DNA, 38 bp, Artificial Sequence
  UU-F1 primer
aaattcctcgtaaaggcggacnnnnnatgattaaatca

SEQ ID NO: 5 - DNA, 38 bp, Artificial Sequence
  UU-F2 primer
gaagcacacaacaaaatggcgnnnnntgtgtatttcac

SEQ ID NO: 6 - DNA, 37 bp, Artificial Sequence
  UU-R1 primer
cataacccccgcccatactaannnnntgaaaacaggg

SEQ ID NO: 7 - DNA, 38 bp, Artificial Sequence
  UU-R2 primer
gctttggctgatgattgcgtagnnnnnatgcaacgtgc

SEQ ID NO: 8 - DNA, 37 bp, Artificial Sequence
  NG-porA-F1 primer
tacgcctgctactttcacgctnnnnngtaatcagatg

SEQ ID NO: 9 - DNA, 37 bp, Artificial Sequence
  NG-porA-R1 primer
caatggatcggtatcactcgcnnnnncgagcaagaac

SEQ ID NO: 10 - DNA, 35 bp, Artificial Sequence
  Ctra-F279 primer
gactcggcttgggaagagctnnnnnggcgtcgtat

SEQ ID NO: 11 - DNA, 37 bp, Artificial Sequence
  Ctra-R626 primer
agccagcactccaatttctgacnnnnngaatatatca

SEQ ID NO: 12 - DNA, 35 bp, Artificial Sequence
  CT-F1 primer
ccttggagcattgtctgggcnnnnnaccaatcccg

SEQ ID NO: 13 - DNA, 35 bp, Artificial Sequence
  CT-R1 primer
tggaccgcatcactcaacaannnnncttgtagatc

SEQ ID NO: 14 - DNA, 37 bp, Artificial Sequence
  CT-F2 primer
tgcaacgggttattcactcccnnnnncattgaaactt

SEQ ID NO: 15 - DNA, 36 bp, Artificial Sequence
  CT-R2 primer
acccataccacaccgctttctnnnnngcctacacgt

SEQ ID NO: 16 - DNA, 40 bp, Artificial Sequence
  CT-ompA-F1 primer
gcttctgggaatacgacctctactnnnnnaaaattggtag

SEQ ID NO: 17 - DNA, 36 bp, Artificial Sequence
  CT-ompA-R1 primer
ccaacactccaagcaaaagtannnnntgtatacagt

SEQ ID NO: 18 - DNA, 35 bp, Artificial Sequence
  CT-ompA-R2 primer
ccacattcccacaaagctgcnnnnnctccaacact

SEQ ID NO: 19 - DNA, 35 bp, Artificial Sequence
  HSV2-glyc-F1 primer
cagcggctcatcatcgaagannnnnccctggagac

SEQ ID NO: 20 - DNA, 35 bp, Artificial Sequence
  HSV2-glyC-R1 primer
tctcctgcgtctgcgtgtgtnnnnnggccggatcg

SEQ ID NO: 21 - DNA, 35 bp, Artificial Sequence
  HSV2-glyC-R2 primer
gcatggtcgcccgtaaactcnnnnntgatggttgg

SEQ ID NO: 22 - DNA, 35 bp, Artificial Sequence
  HSV1-gC-F1 primer
cagcggctgattatcggcgannnnncgcccgcgac

SEQ ID NO: 23 - DNA, 35 bp, Artificial Sequence
  HSV1-gC-R1 primer
gctcgtgcgtctgcgtgtcgnnnnngcccgggtta

SEQ ID NO: 24 - DNA, 35 bp, Artificial Sequence
  HSV1-gC-R2 primer
acatgccggaccccaaattcnnnnntgatggttgg

SEQ ID NO: 25 - DNA, 35 bp, Artificial Sequence
  CA-phr1-F1 primer
tgccgatggtagcaaaggtgnnnnnggtgttgctt

SEQ ID NO: 26 - DNA, 35 bp, Artificial Sequence
  CA-phr1-F2 primer
tcctctggtggaagctccaannnnngatcttcctc

SEQ ID NO: 27 - DNA, 39 bp, Artificial Sequence
  CA-phr1-R1 primer
cgtcctatacaacagaacccttcannnnngttagtcttc

SEQ ID NO: 28 - DNA, 37 bp, Artificial Sequence
  HD-p27-F1 primer
tgcttgcaacaccaaatgatgnnnnncaaaaagtagt

SEQ ID NO: 29 - DNA, 36 bp, Artificial Sequence
  HD-p27-R1 primer
tctacagggttgtttgcaggcnnnnnacaagcagtt

SEQ ID NO: 30 - DNA, 37 bp, Artificial Sequence
  HD-p27-R2 primer
ggttttgttgccattcttggaannnnntgtagtcttc

SEQ ID NO: 31 - DNA, 35 bp, Artificial Sequence
  TV-btub1-F1 primer
gctgaatcctgcgactgcctnnnnngcttccagct

SEQ ID NO: 32 - DNA, 37 bp, Artificial Sequence
  TV-btub1-F2 primer
ctcaactccgaccttcgtaagcnnnnngtcaaccttg

SEQ ID NO: 33 - DNA, 39 bp, Artificial Sequence
  TV-btub1-R1 primer
ggaagtgagcggatgtaaggtaagnnnnncggcgtggat

SEQ ID NO: 34 - DNA, 39 bp, Artificial Sequence
  MG-gyrA-F1 primer
ataatcttcaacatcgtggtggagnnnnngttaaagggc

SEQ ID NO: 35 - DNA, 38 bp, Artificial Sequence
  MG-gyrA-R1 primer
aatctcatcatttccgtgggttnnnnntactgaataca

SEQ ID NO: 36 - DNA, 38 bp, Artificial Sequence
  MG-gyrA-F2 primer
aaaacccacggaaatgatgagannnnnattggttctac

SEQ ID NO: 37 - DNA, 41 bp, Artificial Sequence
  MG-gyrA-R2 primer
ctcccttagcattacgttttgtgannnnntatttatctatg

SEQ ID NO: 38 - DNA, 35 bp, Artificial Sequence
  TP-47-F1 primer
gcgtcatttcaggatttgggnnnnnacggggagat

SEQ ID NO: 39 - DNA, 37 bp, Artificial Sequence
  TP-47-F2 primer
tcacggtatgaagtttgtcccannnnnggttcctcat

SEQ ID NO: 40 - DNA, 39 bp, Artificial Sequence
  TP-47-R1 primer
gcgtcatcaccgtagtagtcgtagnnnnncgtgttgaag

SEQ ID NO: 41 - DNA, 36 bp, Artificial Sequence
  GV-F1 primer
cgctcggttgagtgtggttacnnnnntggaaaacaa

SEQ ID NO: 42 - DNA, 38 bp, Artificial Sequence
  GV-F2 primer
ttggcttgtgttcttggtgtttgnnnnnttgagaactg

SEQ ID NO: 43 - DNA, 35 bp, Artificial Sequence
  GV-R1 primer
aatcccacgaccccgaatacnnnnnacctgacggt

(19) United States
(12) Patent Application Publication
(10) Pub. No.: US 2009/0280486 A1
(43) Pub. Date: Nov. 12, 2009

(54) Oligonucleotides for Detecting Nucleic Acids of Pathogen Causing Sexually Transmitted Diseases

(76) Inventor: Jong Yoon Chun, Seoul (KR)

Correspondence Address: MILES & STOCKBRIDGE PC, 1751 PINNACLE DRIVE, SUITE 500, MCLEAN, VA, 22102-3833, US

(21) Appl. No.: 12/224,179

(22) PCT Filed: Feb. 23, 2007
(86) PCT No.: PCT/KR2007/000940

§ 371 Date: Aug. 20, 2008

(30) Foreign Application Priority Data
Feb. 23, 2006 (KR) 10-2006-0017503

(51) Int. Cl. C12Q 1/68; C07H 21/04

(52) U.S. Cl. 435/6; 536/24.5

(57) ABSTRACT

The present invention relates to oligonucleotides hybridizable with nucleic acids of pathogens causing sexually transmitted diseases, kits comprising them, and processes for amplifying and detecting viral nucleic acids using them. The present oligonucleotides completely overcome problems of false-negative and false-positive products in detection of pathogens causing sexually transmitted diseases using conventional primers and show dramatic workability in multiplex PCR, enabling to simultaneously detect pathogens causing sexually transmitted diseases in a single PCR reaction.

### FIELD OF THE INVENTION

The present invention relates to oligonucleotides hybridizable with nucleic acids of pathogens causing sexually transmitted diseases, kits comprising them, and processes for amplifying and detecting pathogens using them.

### DESCRIPTION OF THE RELATED ART

Sexually transmitted diseases are epidemic diseases caused by pathogens such as diseases bacteria, fungi, viruses, protozoa and parasites, which are mainly transmitted by sexual intercourse.

Syphilis, gonorrhea, soft chancre, granuloma inguinale and lymphogranuloma venereum had been collectedly called as venereal diseases. Thereafter, several sexual contact-causing diseases, gonococcal urethritis, herpes, condyloma accuminatum, pediculosis pubis, trichomoniasis and candidiasis were discovered and reported, an all those caused by sexual intercourse has been collectedly called as sexually transmitted diseases.

Sexually transmitted diseases include life-threatened diseases such as syphilis and AIDS as well as diseases showing slight conditions such as itching, unpleasant feeling and vaginitis. Sexually transmitted diseases showing slight conditions should be treated and their improper treatments are very likely to induce recurrence of diseases, sterility and birth of deformed child.

Generally, the number of pathogens causing sexually transmitted diseases has been more than 30 species, of which representative includes: Mycoplasma hominis, Ureaplasma urealyticum, Neisseria gonorrheae, Chlamydia trachomatis, Herpes simplex virus 1 or 2, Candida albicans, Haemophilus ducreyi, Trichomonas vaginalis, Mycoplasnia genitalium, Treponema pallidum and Gardnella vaginalis.

Sexually transmitted diseases may be conveniently diagnosed and treated according to conditions and symptoms. However, for providing more effective therapy, the type of pathogens has to be detected and identified. Pathogens have been identified merely by clinical manifestation or presentation. To obtain more accurate identification, traditional tests using smear and culture, sugar-using tests, immunofluorescence tests, co-agglutination and monoclonal antibody-using tests have been performed. Currently, tests with detecting pathogen RNA or DNA molecules are studied.

Methods using PCR as tools to detect RNA and DNA molecules have been highlighted in terms of accuracy and convenience. However, PCR methods have serious problems, i.e., production of false-positive results associated with non-specific annealing of primers.

In particular, where multiplex PCR amplification is conducted for detecting simultaneously a plurality of pathogens, conventional primers have restrictions in designing primer sequences and produce much more false-positive results.

Throughout this application, various patents and publications are referenced, and citations are provided in parentheses. The disclosure of these patents and publications in their entities are hereby incorporated by references into this application in order to more fully describe this invention and the state of the art to which this invention pertains.

### DETAILED DESCRIPTION OF THIS INVENTION

The present inventor has made intensive researches to propose a novel approach to detect sexually transmitted disease-causing pathogens with much higher accuracy in a convenient and rapid manner, and as a result discovered that a variety of sexually transmitted disease-causing pathogens are accurately detected using the hybridization oligonucleotides having a unique structure of the dual specificity oligonucleotides developed by the present inventor.

Accordingly, it is an object of this invention to provide an oligonucleotide hybridizable specifically with a nuclei acid molecule of a sexually transmitted disease-causing pathogen.

It is another object of this invention to provide a kit for detecting or amplifying a nuclei acid molecule of a sexually transmitted disease-causing pathogen.

It is still another object of this invention to provide a method for detecting or amplifying a nuclei acid molecule of a sexually transmitted disease-causing pathogen.

Other objects and advantages of the present invention will become apparent from the detailed description to follow taken in conjugation with the appended claims and drawings.

The present invention relates to oligonucleotides to hybridize specifically with nucleic acid molecules of sexually transmitted disease-causing pathogens.

More specifically, the present invention provides an oligonucleotide hybridizable specifically with a nuclei acid molecule of sexually transmitted disease-causing pathogens, which is represented by the following general formula:

5′-Xₚ-Y_{q}-Zᵣ-3′

wherein, Xₚ represents a 5′-high Tₘ specificity portion having a hybridizing nucleotide sequence substantially complementary to a target sequence to hybridize therewith, Y_{q} represents a separation portion comprising at least two universal bases, Zᵣ represents a 3′-low Tₘ specificity portion having a hybridizing nucleotide sequence substantially complementary to a target sequence to hybridize therewith, p, q and r represent the number of nucleotides, and X, Y, and Z are deoxyribonucleotide or ribonucleotide; the Tₘ of the 5′-high Tₘ specificity portion is higher than that of the 3′-low Tₘ specificity portion, the separation portion has the lowest Tₘ in the three portions; the separation portion forms a non base-pairing bubble structure under conditions that the 5′-high Tₘ specificity portion and the 3′-low Tₘ specificity portion are annealed to the target sequence, enabling the 5′-high Tₘ specificity portion to separate from the 3′-low Tₘ specificity portion in terms of hybridization specificity to the target sequence, whereby the hybridization specificity of the oligonucleotide is determined dually by the 5′-high Tₘ specificity portion and the 3′-low Tₘ specificity portion such that the overall hybridization specificity of the oligonucleotide is enhanced; wherein the sexually transmitted disease-causing pathogen is Mycoplasma hominis, Ureaplasma urealyticum, Neisseria gonorrheae, Chlamydia trachomatis, Herpes simplex virus-1 or 2, Candida albicans, Haemophilus ducreyi, Trichomonas vaginalis, Mycoplasma genitalium, Treponema pallidum or Gardnella vaginalis; and wherein the target sequence is the genome nucleic acid molecule of the sexually transmitted disease-causing pathogen.

The present invention is directed to oligonucleotides hybridizable with a nucleic acid molecule of Mycoplasma hominis, Ureaplasma urealyticum, Neisseria gonorrheae, Chlamydia trachomatis, Herpes simplex virus-1 or 2, Candida albicans, Haemophilus ducreyi, Trichomonas vaginalis, Mycoplasma genitalium, Treponema pallidum or Gardnella vaginalis.

The term used herein “nucleic acid molecule of pathogens” refers to not only genomes of pathogens but also any RNA or DNA molecule (e.g., cDNA) derived from genomes. Furthermore, the term used herein “nucleic acid molecule of pathogens” is intended to encompass plasmids in pathogens.

The term used “hybridization” herein refers to the formation of a double-stranded nucleic acid by base-paring of complementary single stranded nucleic acids. Hybridization may occur between single stranded nucleic acids with some mismatched sequences as well as between single stranded nucleic acids with perfect complementarity. The complementarity for hybridization depends on hybridization conditions, in particular, temperature. Generally, as the hybridization temperature becomes higher, only perfectly complementary sequences are likely to be hybridized; in contrast, as the hybridization temperature becomes lower, hybridization may occurs between single stranded nucleic acids with some mismatched sequences. As the hybridization temperature becomes lower, the mismatch occurs with higher frequency.

The fundamental structure of oligonucleotides of this invention has been first proposed by the present inventor and called as a structure with dual specificity. Therefore, oligonucleotides having such structure are named as dual specificity oligonucleotides (DSO). The DSO embodies a novel concept and its hybridization is dually determined by the 5′-high Tₘ specificity portion and the 3′-low Tₘ specificity portion separated by the separation portion, exhibiting dramatically enhanced specificity (see PCT/KR2005/001206).

According to a preferred embodiment, the universal base in the separation portion is selected from the group consisting of deoxyinosine, inosine, 7-deaza-2′-deoxyinosine, 2-aza-2′-deoxyinosine, 2′-OMe inosine, 2′-F inosine, deoxy 3-nitropyrrole, 3-nitropyrrole, 2′-OMe 3-nitropyrrole, 2′-F 3-nitropyrrole, 1-(2′-deoxy-beta-D-ribofuranosyl)-3-nitropyrrole, deoxy 5-nitroindole, 5-nitroindole, 2′-OMe 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′O-methoxyethyl nebularine, 2′-O-methoxyethyl 5-nitroindole, 2′-O-methoxyethyl 4-nitro-benzimidazole, 2′-O-methoxyethyl 3-nitropyrrole, and combinations thereof. More preferably, the universal base or non-discriminatory base analog is deoxyinosine, 1-(2′-deoxy-beta-D-ribofuranosyl)-3-nitropyrrole or 5-nitroindole, most preferably, deoxyinosine.

It is preferable that the separation portion comprises contiguous nucleotides having universal bases, preferably, deoxyinosine.

Preferably, the 5′-high Tₘ specificity portion is longer than the 3′-low Tₘ specificity portion. The 5′-high Tₘ specificity portion is preferably 15-40 nucleotides, more preferably 15-25 nucleotides in length.

It is preferable that the 3′-low Tₘ specificity portion is 3-15 nucleotides, more preferably 6-13 nucleotides in length.

The separation portion is preferably 3-10 nucleotides, more preferably 4-8 nucleotides, most preferably 5-7 nucleotides in length.

According to a preferred embodiment, the Tₘ of the 5′-high Tₘ specificity portion ranges from 40° C. to 80° C. The Tₘ of the 3′-low Tₘ specificity portion ranges preferably from 10° C. to 40° C. It is preferable that the Tₘ of the separation portion ranges from 3° C. to 15° C.

According to a preferred embodiment, the pathogen-specific oligonucleotide of this invention has a sequence specific to a pathogen species and common (i.e., conserved sequences) in various isolates or strains of a pathogen species, and has the structure of the dual specificity oligonucleotide (DSO). The main reference sequences for preparing oligonucleotides of this invention are conserved sequences selected by searching publicly-known nucleotide sequences of isolates or stains of a pathogen species, which is specific to the pathogen species. Among the selected sequences, a sequence suitable to design primers or probes having the DSO structure is then determined.

According to a preferred embodiment, the target nucleotide sequence hybridized with the oligonucleotides of the present invention includes a nucleotide sequence of a gene in a genome of pathogens, rRNA-coding sequence, an internal sequence between rRNA-coding sequences and a nucleotide sequence in plasmid.

According to a preferred embodiment, the target nucleotide sequence may be selected on the basis of publicly-known nucleotide sequences. For example, the target nucleotide sequence may be selected with referring to sequences described in the following data bases: for Mycoplasma hominis, GenBank accession Nos.: AJ243692, AY879770, AF443617, Z98055, Z27121, AJ132792, AJ005058 and AY738737; for Ureaplasma urealyticum, GenBank accession Nos.: AF085729, U50459, L20329, AY641822, X51315, Z34275 and AF272621; for Neisseria gonorrheae, GenBank accession Nos.: AJ223447, AE004969, M10316 and U20374; for Chlamydia trachomatis, GenBank accession Nos.: M19487, AE001273 and CP000051; for Herpes simplex virus 2, GenBank accession No.: Z86099; for Herpes simplex virus 1, GenBank accession Nos.: AJ421498 and X14112; for Candida albicans, GenBank accession Nos.: M90812 and AP006852; for Haciemophilus ducreyi, GenBank accession Nos.: AF087639, AY434675 and AE017143; for Trichomionas vaginalis, GenBank accession No.: L05468; for Mycoplasma genitalium, GenBank accession Nos.: L43967, AY386817 and AY816341; for Treponema pallidum, GenBank accession Nos.: M88769, AE000520, X61228 and X54111; and for Gardnella vaginalis, GenBank accession Nos.: L08167, M58744 and M85116.

According to a preferred embodiment, the suitable target sequences of pathogens are indicated as used sequences in Tables 1a-1b.

According to a preferred embodiment, the sequence of the hybridizing portions in the present oligonucleotide is designed to be specifically hybridized with the target sequence described above.

Most preferably, the oligonucleotide of this invention comprises the nucleotide sequence selected from the group consisting of SEQ ID NOs:1-43 specifically hybridized with the target sequence of pathogens described above.

The nucleotide sequences of SEQ ID NOs:1-43 and their target pathogens are summarized in Tables 1a-1b.

TABLE 1a

| SEQ | | | | |
|---|---|---|---|---|
| ID | | | | Used target |
| NO | Pathogens | Name | Primer sequence (5′ → 3′) | sequence |
| 1 | Mycoplasma | MH- | TGC TCC AGC TAA AAG CGA AGG IIIII | gap |
| | hominiss | gap-F1 | AA CAG TTG TT | |
| 2 | | MH- | ACT GTT TAG CTC CTA TTG CCA ACG | |
| | | gap-F2 | IIIII GAA AAA AAC TT | |
| 3 | | MH- | GCC TGC TTT TGC ACC AAT AAT A | |
| | | gap-R2 | IIIII TGA TAC AAT TG | |
| 4 | Ureaplasma | UU-F1 | AAA TTC CTC GTA AAG GCG GAC IIIII | ureG and |
| | urealyticum | | ATG ATT AAA TCA | ureD |
| 5 | | UU-F2 | GAA GCA CAC AAC AAA ATG GCG | |
| | | | IIIII TGT GTA TTT CAC | |
| 6 | | UU-R1 | CAT AAC CCC CGC CCA TAC TAA IIIII | |
| | | | TGA AAA CAG GG | |
| 7 | | UU-R2 | GCT TTG GCT GAT GAT TGC GTA G | |
| | | | IIIII ATG CAA CGT GC | |
| 8 | Neisseria | NG | TAC GCC TGC TAC TTT CAC GCT IIIII | porA |
| | gonorrheae | porA- | GTA ATC AGA TG | pseudogene |
| | | Fl | | |
| 9 | | NG | CAA TGG ATC GGT ATC ACT CGC IIIII | |
| | | porA- | CGA GCA AGA AC | |
| | | R1 | | |
| 10 | Chlamydia | Ctra- | GACTCGGCTTGGGAAGAGCT IIIII | pCTT1 |
| | trachomatis | F279 | GGCGTCGTAT | |
| 11 | | Ctra- | AGCCAGCACTCCAATTTCTGAC IIIII | |
| | | R626 | GAATATATCA | |
| 12 | | CT-F1 | CCT TGG AGC ATT GTC TGG GC IIIII | |
| | | | ACC AAT CCC G | |
| 13 | | CT-R1 | TGG ACC GCA TCA CTC AAC AA IIIII | |
| | | | CTT GTA GAT C | |
| 14 | | CT-F2 | TGC AAC GGG TTA TTC ACT CCC IIIII | |
| | | | CAT TGA AAC TT | |
| 15 | | CT-R2 | ACC CAT ACC ACA CCG CTT TCT IIIII | |
| | | | GCC TAC ACG T | |
| 16 | | CT- | GCTTCTGGGAATACGACCTCTACT | ompA |
| | | ompA- | IIIII AAAATTGGTAG | |
| | | F1 | | |
| 17 | | CT- | CCAACACTCCAAGCAAAAGTA IIIII | |
| | | ompA- | TGTATACAGT | |
| | | R1 | | |
| 18 | | CT- | CCACATTCCCACAAAGCTGC IIIII | |
| | | ompA- | CTCCAACACT | |
| | | R2 | | |
| 19 | Herpes | HSV2- | CAG CGG CTC ATC ATC GAA GA IIIII | glycoprotein |
| | simplex virus- | glyC-F1 | CCC TGG AGA C | C |
| 20 | 2 | HSV2- | TCT CCT GCG TCT GCG TGT GT IIIII | |
| | | glyC- | GGC CGG ATC G | |
| | | R1 | | |
| 21 | | HSV2- | GCA TGG TCG CCC GTA AAC TC IIIII | |
| | | glyC- | TGA TGG TTG G | |
| | | R2 | | |

TABLE 1b

| | | | | |
|---|---|---|---|---|
| 22 | Herpes | HSV1- | CAGCGGCTGATTATCGGCGA IIIII | gC-1 |
| | simplex | gC-F1 | CGCCCGCGAC | |
| 23 | virus-1 | HSV1- | GCTCGTGCGTCTGCGTGTCG IIIII | |
| | | gC-R1 | GCCCGGGTTA | |
| 24 | | HSV1- | ACATGCCGGACCCCAAATTC IIIII | |
| | | gC-R2 | TGATGGTTGG | |
| 25 | Candida | CA- | TGCCGATGGTAGCAAAGGTG IIIII | pH |
| | albicans | phr1-F1 | GGTGTTGCTT | responsive |
| 26 | | CA- | TCCTCTGGTGGAAGCTCCAA IIIII | protein |
| | | phr1-F2 | GATCTTCCTC | |
| 27 | | CA- | CGTCCTATACAACAGAACCCTTCA IIIII | |
| | | phr1-R1 | GTTAGTCTTC | |
| 28 | Haemophilus | HD- | TGCTTGCAACACCAAATGATG IIIII | P27 |
| | ducreyi | p27-F1 | CAAAAAGTAGT | |
| 29 | | HD- | TCTACAGGGTITGTTTGCAGGC IIIII | |
| | | p27-R1 | ACAAGCAGTT | |
| 30 | | HD- | GGTTTTGTTGCCATTCTTGGAA IIIII | |
| | | p27-R2 | TGTAGTCTTC | |
| 31 | Trichomonas | TV- | GCTGAATCCTGCGACTGCCT IIIII | beta-tubulin |
| | vaginalis | btub1- | GCTTCCAGCT | 1 |
| | | F1 | | |
| 32 | | TV- | CTCAACTCCGACCTTCGTAAGC IIIII | |
| | | btub1- | GTCAACCTTG | |
| | | F2 | | |
| 33 | | TV- | GGAAGTGAGCGGATGTAAGGTAAG | |
| | | btub1- | IIIII CGGCGTGGAT | |
| | | R1 | | |
| 34 | Mycplasma | MG- | ATAATCTTCAACATCGTGGTGGAG IIIII | gyrA |
| | genitalium | gyrA-F1 | GTTAAAGGGC | |
| 35 | | MG- | AATCTCATCATTTCCGTGGGTT IIIII | |
| | | gyrA-R1 | TACTGAATACA | |
| 36 | | MG- | AAAACCCACGGAAATGATGAGA IIIII | |
| | | gyrA-F2 | ATTGGTTCTAC | |
| 37 | | MG- | CTCCCTTAGCATTACGTTTTGTGA IIIII | |
| | | gyrA-R2 | TATTTATCTATG | |
| 38 | Treponema | TP-47- | GCGTCATTTCAGGATTTGGG IIIII | 47 kd |
| | pallidum | F1 | ACGGGGAGAT | antigen |
| 39 | | TP-47- | TCACGGTATGAAGTTTGTCCCA IIIII | |
| | | F2 | GGTTCCTCAT | |
| 40 | | TP-47- | GCGTCATCACCGTAGTAGTCGTAG IIIII | |
| | | R1 | CGTGTTGAAG | |
| 41 | Gardnella | GV-F1 | CGCTCGGTTGAGTGTGGTTAC IIIII | 16S & 23S |
| | vaginali | | TGGAAAACAA | rRNA |
| | | GV-F2 | TTGGCTTGTGTTCTTGGTGTTTG IIIII | (internal |
| | | | TTGAGAACTG | transcribed |
| | | GV-R1 | AATCCCACGACCCCGAATAC IIIII | spacer) |
| | | | ACCTGACGGT | |

*I: deoxyinosine

The present oligonucleotides embodying the DSO structure with the conserved sequences completely eliminate false-positive results and backgrounds associated with existing methods using conventional primers for detecting respiratory viruses.

The oligonucleotide of this invention comprises not only the nucleotide sequence selected from the group consisting of SEQ ID NOs:1-43 but also a complementary sequence to and a substantially identical nucleotide sequence to that. The term used herein “substantially identical nucleotide sequence” refers to a nucleotide sequence having some deletions, additions and/or substitutions in the nucleotide sequence selected from the group consisting of SEQ ID NOs:1-43. Such nucleotide changes are permissible, so long as the oligonucleotide can be specifically hybridized with a target sequence. It will be appreciated under the doctrine of equivalency that these substantially identical nucleotide sequences fall within the scope of claims.

In one embodiment of this invention, the oliogonucleotides of this invention serve as probes for detecting target pathogen nucleic acids. The term used herein “probe” means a single-stranded nucleic acid molecule comprising a portion or portions that are substantially complementary to a target nucleotide sequence. Some modifications in the oliogonucleotides of this invention can be made unless the modifications abolish the advantages of the oliogonucleotides. Such modifications, i.e., labels linking to the oliogonucleotides generate a signal to detect hybridization. Suitable labels include fluorophores, chromophores, chemiluminescers, magnetic particles, radioisotopes, mass labels, electron dense particles, enzymes, cofactors, substrates for enzymes and haptens having specific binding partners, e.g., an antibody, streptavidin, biotin, digoxigenin and chelating group, but not limited to. The labels generate signal detectable by fluorescence, radioactivity, measurement of color development, mass measurement, X-ray diffraction or absorption, magnetic force, enzymatic activity, mass analysis, binding affinity, high frequency hybridization or nanocrystal. The labels may be linked to the 5′-end, 3′-end or inner portions of the oligonucleotides. Labeling may be performed directly (e.g., with dyes) or indirectly (e.g., with biotin, digoxin, alkaline phosphatase or horseradish peroxidase).

According to a preferred embodiment, the oligonucleotides may be immobilized on a solid substrate (nitrocellulose membrane, nylon filter, glass plate, silicon wafer and fluorocarbon support) to fabricate microarray. In microarray, the present oligonucleotides serve as hybridizable array elements. The immobilization on solid substrates may occur through chemical binding or covalent binding by ultra-violet radiation. In an embodiment of this invention, the oligonucleotides are bound to a glass surface modified to contain epoxy compounds or aldehyde groups or to a polylysin-coated surface. Furthermore, the oligonucleotides are bound to a substrate through linkers (e.g. ethylene glycol oligomer and diamine).

According to one embodiment of this invention, the oliogonucleotides of this invention serve as primers for detecting target pathogen nucleic acids by amplification. The term “primer” as used herein refers to an oligonucleotide, which is capable of acting as a point of initiation of synthesis when placed under conditions in which synthesis of primer extension product which is complementary to a nucleic acid strand (template) is induced, i.e., in the presence of nucleotides and an agent for polymerization, such as DNA polymerase, and at a suitable temperature and pH. The primer is preferably single stranded for maximum efficiency in amplification. Preferably, the primer is an oligodeoxyribonucleotide. The primer of this invention can be comprised of naturally occurring dNMP (i.e., dAMP, dGM, dCMP and dTMP), modified nucleotide, or non-natural nucleotide. The primer can also include ribonucleotides. For instance, the oligonucleotides of this invention For example, the oligonucleotide of this invention may include nucleotides with backbone modifications such as peptide nucleic acid (PNA) (M. Egholm et al., Nature, 365:566-568 (1993)), phosphorothioate DNA, phosphorodithioate DNA, phosphoramidate DNA, amide-linked DNA, MMI-linked DNA, 2′-O-methyl RNA, alpha-DNA and methylphosphonate DNA, nucleotides with sugar modifications such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and nucleotides having base modifications such as C-5 substituted pyrimidines (substituents including fluoro-, bromo-, chloro-, iodo-, methyl-, ethyl-, vinyl-, formyl-, ethynyl-, propynyl-, alkynyl-, thiazolyl-, imidazolyl-, pyridyl-), 7-deazapurines with C-7 substituents (substituents including fluoro-, bromo-, chloro-, iodo-, methyl-, ethyl-, vinyl-, formyl-, alkynyl-, alkenyl-, thiazolyl-, imidazolyl-, pyridyl-), inosine, and diaminopurine.

The sequences of the primers may comprise some mismatches, so long as they can be hybridized with templates and serve as primers.

In one embodiment of this invention, the nucleic acid amplification of target pathogen nucleic acids using the oligonucleotides of this invention is used to detect target pathogens.

In another aspect of this invention, there is provided an oligonucleotide pair hybridizable specifically with a nuclei acid molecule of a sexually transmitted disease-causing pathogen.

The preferable oligonucleotide pairs of the present invention are: for Mycoplasma hominis, SEQ ID NOs:1 and 3, or SEQ ID NOs:2 and 3; for Ureaplasma urealyticum, SEQ ID NOs:4 and 6, SEQ ID NOs:4 and 7, SEQ ID NOs:5 and 6, SEQ ID NOs:5 and 7; for Neisseria gonorrheae, SEQ ID NOs:8 and 9; for Chlamydia trachomatis, SEQ ID NOs:10 and 11, SEQ ID NOs:12 and 13, SEQ ID NOs:14 and 15, SEQ ID NOs:16 and 17, or SEQ ID NOs:16 and 18; for Herpes simplex virus-2, SEQ ID NOs:19 and 20, or SEQ ID NOs:19 and 21; for Herpes simplex virus-1, SEQ ID NOs:22 and 23, or SEQ ID NOs:22 and 24; for Candida albicans, SEQ ID NOs:25 and 27, or SEQ ID NOs:26 and 27; for Haemophilus ducreyi, SEQ ID NOs:28 and 29, or SEQ ID NOs:28 and 30; for Trichomonas vaginalis, SEQ ID NOs:31 and 33, or SEQ ID NOs:32 and 33; for Mycoplasma genitalium, SEQ ID NOs:34 and 35, or SEQ ID NOs:36 and 37; for Treponema pallidum, SEQ ID NOs:38 and 40, or SEQ ID NOs:39 and 40; for Gardnella vaginalis, SEQ ID NOs:41 and 43, or SEQ ID NOs:42 and 43.

Since the oligonucleotide pair comprises the oligonucleotides of this invention described hereinabove, the common descriptions between them are omitted in order to avoid undue redundancy leading to the complexity of this specification.

Preferably, the oligonucleotide pair of the present invention is used as a primer pair (forward and reverse primers) for detecting sexually transmitted disease-causing pathogens.

In still another aspect of this invention, there is provided an oligonucleotide set hybridizable with nucleic acid molecules of sexually transmitted diseases-causing pathogens comprising a least two oligonucleotide pairs described above.

Since the oligonucleotide set comprises the oligonucleotides of this invention described hereinabove, the common descriptions between them are omitted in order to avoid undue redundancy leading to the complexity of this specification.

The oligonucleotide set is useful not only as a probe set in detecting sexually transmitted diseases-causing pathogens but also as a primer set in amplifying nucleic acids of sexually transmitted diseases-causing pathogens. If necessary, several types of the present oligonucleotides may be simultaneously used. Such simultaneous application does not influence adversely on either each result or overall results.

According to a preferred embodiment, the oligonucleotide set is used as primer sets for multiplex PCR.

According to a preferred embodiment, the oligonucleotide set is composed of SEQ ID NOs: 2 and 3; SEQ ID NOs: 5 and 6; SEQ ID NOs: 8 and 9; SEQ ID NOs: 10 and 11; and SEQ ID NOs: 19 and 20 (SEQ ID NOs: 22 and 23).

According to a preferred embodiment, the oligonucleotide set is composed of SEQ ID NOs: 26 and 27; SEQ ID NOs: 28 and 29; SEQ ID NOs: 31 and 33; SEQ ID NOs: 34 and 35; SEQ ID NOs: 39 and 40; and SEQ ID NOs: 42 and 43.

The embodiments for primer sets described hereinabove are non-limiting embodiments and therefore a multitude of primer combinations can be prepared in considering the sizes of PCR products.

In further aspect of this invention, there is provided a kit for detecting a sexually transmitted disease-causing pathogen or a kit for amplifying a target nucleotide sequence of a sexually transmitted disease-causing pathogen comprising the oligonucleotides, oligonucleotide pairs or oligonucleotide sets of this invention.

Since the kit comprises the oligonucleotides of this invention described hereinabove, the common descriptions between them are omitted in order to avoid undue redundancy leading to the complexity of this specification.

The present kits may optionally include the reagents required for performing PCR reactions such as buffers, DNA polymerase, DNA polymerase cofactors, and deoxyribonucleotide-5-triphosphates. Optionally, the kits may also include various polynucleotide molecules, reverse transcriptase, various buffers and reagents, and antibodies that inhibit DNA polymerase activity.

The kits may also include reagents necessary for performing positive and negative control reactions. Optimal amounts of reagents to be used in a given reaction can be readily determined by the skilled artisan having the benefit of the current disclosure. The kits, typically, are adapted to contain in separate packaging or compartments the constituents afore-described.

In still further aspect of this invention, there is provided a method for detecting a sexually transmitted disease-causing pathogen, which comprises hybridizing the oligonucleotide, oligonucleotide pair or oligonucleotide set of this invention with a nucleic acid molecule of a sexually transmitted disease-causing pathogen.

In another aspect of this invention, there is provided a method for amplifying a nucleic acid molecule of a sexually transmitted disease-causing pathogen, which comprises hybridizing the oligonucleotide, oligonucleotide pair or oligonucleotide set of this invention with a nucleic acid molecule of a sexually transmitted disease-causing pathogen.

Suitable hybridization conditions may be routinely determined by optimization procedures. Conditions such as temperature, concentration of components, hybridization and washing times, buffer components, and their pH and ionic strength may be varied depending on various factors, including the length and GC content of oligonucleotide and target nucleotide sequence. The detailed conditions for hybridization can be found in Joseph Sambrook, et al., Molecular Cloning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999).

According to a preferred embodiment, the hybridization is performed at temperature of 40-70° C., more preferably, 45-68° C., most preferably 50-65° C.

The present method for amplifying nucleic acids of sexually transmitted disease-causing pathogens may be carried out according to a variety of conventional amplification processes.

The present method for amplifying nucleic acids may be applied to the amplification of nucleic acids of any sexually transmitted disease-causing pathogens. The nucleic acid molecule may be either DNA or RNA. The molecule may be in either a double-stranded or single-stranded form. Where the nucleic acid as starting material is double-stranded, it is preferred to render the two strands into a single-stranded or partially single-stranded form. Methods known to separate strands includes, but not limited to, heating, alkali, formamide, urea and glycoxal treatment, enzymatic methods (e.g., helicase action), and binding proteins. For instance, strand separation can be achieved by heating at temperature ranging from 80° C. to 105° C. General methods for accomplishing this treatment are provided by Joseph Sambrook, et al., Molecular Cloning, A Laboratory Manual, Cold Spring Harbor Laboratory Press, Cold Spring Harbor, N.Y. (2001).

Where a mRNA is employed as starting material, a reverse transcription step is necessary prior to performing annealing step, details of which are found in Joseph Sambrook, et al., Molecular Cloning, A Laboratory Manual, Cold Spring Harbor Laboratory Press, Cold Spring Harbor, N.Y. (2001); and Noonan, K. F. et al., Nucleic Acids Res. 16:10366 (1988)). For reverse transcription, an oligonucleotide dT primer hybridizable to poly A tail of mRNA is used. The oligonucleotide dT primer is comprised of dTMPs, one or more of which may be replaced with other dNMPs so long as the dT primer can serve as primer. Reverse transcription can be done with reverse transcriptase that has RNase H activity. If one uses an enzyme having RNase H activity, it may be possible to omit a separate RNase H digestion step by carefully choosing the reaction conditions.

The primer used for the present invention is hybridized or annealed to a site on the template such that double-stranded structure is formed. Conditions of nucleic acid annealing suitable for forming such double stranded structures are described by Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985).

A variety of DNA polymerases can be used in the amplification step of the present methods, which includes “Klenow” fragment of E. coli DNA polymerase I, a thermostable DNA polymerase, and bacteriophage T7 DNA polymerase. Preferably, the polymerase is a thermostable DNA polymerase which may be obtained from a variety of bacterial species, including Thermus aquaticus (Taq), Thermus thermophilus (Tth), Thermus filiformis, Thermis flavus, Thermococcus literalis, and Pyrococcus furiosus (Pfu). Many of these polymerases may be isolated from bacterium itself or obtained commercially. Polymerase to be used with the subject invention can also be obtained from cells which express high levels of the cloned genes encoding the polymerase.

When a polymerization reaction is being conducted, it is preferable to provide the components required for such reaction in excess in the reaction vessel. Excess in reference to components of the extension reaction refers to an amount of each component such that the ability to achieve the desired extension is not substantially limited by the concentration of that component. It is desirable to provide to the reaction mixture an amount of required cofactors such as Mg²⁺, dATP, dCTP, dGTP, and dTTP in sufficient quantity to support the degree of the extension desired.

All of the enzymes used in this amplification reaction may be active under the same reaction conditions. Indeed, buffers exist in which all enzymes are near their optimal reaction conditions. Therefore, the amplification process of the present invention can be done in a single reaction volume without any change of conditions such as addition of reactants.

Annealing or hybridization in the present method is performed under stringent conditions that allow for specific binding between the primer and the template nucleic acid (at this time, the separation portion cannot be annealed to the template nucleic acid). Such stringent conditions for annealing will be sequence-dependent and varied depending on environmental parameters. Preferably, the annealing temperature ranges from 40° C. to 70° C., more preferably from 45° C. to 68° C., most preferably from 50° C. to 65° C.

In the most preferable embodiment, the amplification is performed in accordance with PCR which is disclosed in U.S. Pat. Nos. 4,683,195, 4,683,202, and 4,800,159.

The present methods may be combined with many other processes known in the art to achieve a specific aim. For example, the isolation (or purification) of amplified product may follow the second-stage amplification. This can be accomplished by gel electrophoresis, column chromatography, affinity chromatography or hybridization. In addition, the amplified product of this invention may be inserted into suitable vehicle for cloning. Furthermore, the amplified product of this invention may be expressed in suitable host harboring expression vector. In order to express the amplified product, one would prepare an expression vector that carries the amplified product under the control of, or operatively linked to a promoter. Many standard techniques are available to construct expression vectors containing the amplified product and transcriptional/translational/control sequences in order to achieve protein or peptide expression in a variety of host-expression systems. The promoter used for prokaryotic host includes, but not limited to, pLλ promoter, trp promoter, lac promoter and T7 promoter. The promoter used for eukaryotic host includes, but not limited to, metallothionein promoter, adenovirus late promoter, vaccinia virus 7.5K promoter and the promoters derived from polyoma, adenovirus 2, simian virus 40 and cytomegalo virus. Certain examples of prokaryotic hosts are E. coli, Bacillus subtilis, and other enterobacteriaceae such as Salmonella typhimurium, Serratia marcescens, and various Pseudomonas species. In addition to microorganisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, tobacco mosaic virus) or transformed with recombinant plasmid expression vectors (e.g., Ti plasmid) containing one or more coding sequences. The expressed polypeptide from the amplified product may be generally purified with a variety of purposes in accordance with the method known in the art.

According to a preferred embodiment, the present method is carried out according to multiplex PCR. According to a conventional multiplex PCR, the results obtained with multiplex PCR are frequently complicated by the artifacts of the amplification procedure. These include “false-negative” results due to reaction failure and “false-positive” results such as the amplification of spurious products, which may be caused by annealing of the primers to sequences which are related to but distinct from the true recognition sequences. Therefore, elaborate optimization steps of multiplex PCR are conducted to reduce such false results; however, the optimization of the reaction conditions for multiplex PCR may become labor-intensive and time-consuming and unsuccessful. The present method amplifies simultaneous a variety of nucleic acid molecules of sexually transmitted disease-causing pathogens with no false results in a single PCR reaction to completely overcome shortcomings associated with conventional multiplex PCR.

The advantages of this invention are will be described as follows:

(a) the oligonucleotides of the present invention ensure to completely overcome problems of false-negative and false-positive products because of their higher specificity; and

(b) the present oligonucleotides exhibit dramatic workability in multiplex PCR, enabling to simultaneously detect various sexually transmitted disease-causing pathogens in a single PCR reaction.

### BRIEF DESCRIPTION OF THE DRAWINGS

FIG. 1 represents the results of multiplex PCR amplifications using the primer set I of the present invention and pathogen samples obtained from 12 patients. 1-24 (the number of patients), N: negative control (containing only internal control), Internal: internal control, MH: Mycoplasma hominis, CT: Chlamydia trachomatis, HSV-2: Herpes simplex virus-2, NG: Neisseria gonorrheae, UU: Ureaplasma urealyticum.

FIG. 2 represents the results of multiplex PCR amplifications using the primer set II of the present invention and pathogen samples obtained from 12 patients. 1-24 (the number of patients), N: negative control (containing only internal control), Internal: internal control, TV: Trichomonas vaginalis, GV: Gardnella vaginalis, MG: Mycoplasma genitalium, TP: Treponema pallidum, HD: Haemophilus ducreyi, CA: Candida albicans.

The present invention will now be described in further detail by examples. It would be obvious to those skilled in the art that these examples are intended to be more concretely illustrative and the scope of the present invention as set forth in the appended claims is not limited to or by the examples.

### EXAMPLE

### Example I

### Primer Design and Preparation

Conserved sequences were discovered by comparing the nucleotide sequences of isolates or strains in a target pathogen species. The conserved sequences were specific in the target pathogen species and distinctly different from other pathogen species.

### Example I-1

### Primers for Amplifying Nucleic Acids of Mycoplasma hominis

In the selected sequence of the gap gene of Mycoplasma hominis, a suitable sequence to prepare DSO (dual specificity oligonucleotide) primers of this invention was determined. Forward and reverse primers were designed using the determined sequence. The symbol “I” denotes deoxyinosine in the following sequences.

MH-gap-F1

(SEQ ID NO: 1)

TGC TCC AGC TAA AAG CGA AGG IIIII AAA CAG TTG TT
MH-gap-F2

(SEQ ID NO: 2)

ACT GTT TAG CTC CTA TTG CCA ACG IIIII GAA AAA AAC
TT
MH-gap-R2

(SEQ ID NO: 3)

GCC TGC TTT TGC ACC AAT AAT A IIIII TGA TAC AAT TG

### Example I-2

### Primers for Amplifying Nucleic Acids of Ureaplasma urealyticum

In the selected sequence of the ureG and ureD genes of Ureaplasma urealyticum, a suitable sequence to prepare DSO (dual specificity oligonucleotide) primers of this invention was determined. Forward and reverse primers were designed using the determined sequence. The symbol “I” denotes deoxyinosine in the following sequences.

UU-F1

(SEQ ID NO: 4)

AAA TTC CTC GTA AAG GCG GAC IIIII ATG ATT AAA TCA
UU-F2

(SEQ ID NO: 5)

GAA GCA CAC AAC AAA ATG GCG IIIII TGT GTA TTT CAC
UU-R1

(SEQ ID NO: 6)

CAT AAC CCC CGC CCA TAC TAA IIIII TGA AAA CAG GG
UU-R2

(SEQ ID NO: 7)

GCT TTG GCT GAT GAT TGC GTA G IIIII ATG CAA CGT GC

### Example I-3

Primers for Amplifying Nucleic Acids of Neisseria gonorrheae

In the selected sequence of the porA pseudogene of Neisseria gonorrheae, a suitable sequence to prepare DSO (dual specificity oligonucleotide) primers of this invention was determined. Forward and reverse primers were designed using the determined sequence. The symbol “I” denotes deoxyinosine in the following sequences.

NG-porA-F1

(SEQ ID NO: 8)

TAC GCC TGC TAC TTT CAC GCT IIIII GTA ATC AGA TG
NG-porA-R1

(SEQ ID NO: 9)

CAA TGG ATC GGT ATC ACT CGC IIIII CGA GCA AGA AC

### Example I-4

### Primers for Amplifying Nucleic Acids of Chlamydia trachomatis

In the selected sequence of the pCTT1 or ompA gene of Chlamydia trachomatis, a suitable sequence to prepare DSO (dual specificity oligonucleotide) primers of this invention was determined. Forward and reverse primers were designed using the determined sequence. The symbol “I” denotes deoxyinosine in the following sequences.

Ctra-F279

(SEQ ID NO: 10)

GACTCGGCTTGGGAAGAGCT IIIII GGCGTCGTAT
Ctra-R626

(SEQ ID NO: 11)

AGCCAGCACTCCAATTTCTGAC IIIII GAATATATCA
CT-F1

(SEQ ID NO: 12)

CCT TGG AGC ATT GTC TGG GC IIIII ACC AAT CCC G
CT-R1

(SEQ ID NO: 13)

TGG ACC GCA TCA CTC AAC AA IIIII CTT GTA GAT C
CT-F2

(SEQ ID NO: 14)

TGC AAC GGG TTA TTC ACT CCC IIIII CAT TGA AAC TT
CT-R2

(SEQ ID NO: 15)

ACC CAT ACC ACA CCG CTT TCT IIIII GCC TAC ACG T
CT-ompA-F1

(SEQ ID NO: 16)

GCTTCTGGGAATACGACCTCTACT IIIII AAAATTGGTAG
CT-ompA-R1

(SEQ ID NO: 17)

CCAACACTCCAAGCAAAAGTA IIIII TGTATACAGT
CT-ompA-R2

(SEQ ID NO: 18)

CCACATTCCCACAAAGCTGC IIIII CTCCAACACT

### Example I-5

### Primers for Amplifying Nucleic Acids of Herpes Simplex Virus-2

In the selected sequence of the glycoprotein C gene of Herpes simplex virus-2, a suitable sequence to prepare DSO (dual specificity oligonucleotide) primers of this invention was determined. Forward and reverse primers were designed using the determined sequence. The symbol “I” denotes deoxyinosine in the following sequences.

HSV2-glyC-F1

(SEQ ID NO: 19)

CAG CGG CTC ATC ATC GAA GA IIIII CCC TGG AGA C
HSV2-glyC-R1

(SEQ ID NO: 20)

TCT CCT GCG TCT GCG TGT GT IIIII GGC CGG ATC G
HSV2-glyC-R2

(SEQ ID NO: 21)

GCA TGG TCG CCC GTA AAC TC IIIII TGA TGG TTG G

### Example I-6

### Primers for Amplifying Nucleic Acids of Herpes Simplex Virus-1

In the selected sequence of the gC-1 sequence of Herpes simplex virus-1, a suitable sequence to prepare DSO (dual specificity oligonucleotide) primers of this invention was determined. Forward and reverse primers were designed using the determined sequence. The symbol “I” denotes deoxyinosine in the following sequences.

HSV1-gC-F1

(SEQ ID NO: 22)

CAGCGGCTGATTATCGGCGA IIIII CGCCCGCGAC
HSV1-gC-R1

(SEQ ID NO: 23)

GCTCGTGCGTCTGCGTGTCG IIIII GCCCGGGTTA
HSV1-gC-R2

(SEQ ID NO: 24)

ACATGCCGGACCCCAAATTC IIIII TGATGGTTGG

### Example I-7

### Primers for Amplifying Nucleic Acids of Candida albicans

In the selected sequence of the gene encoding the pH-responsive protein of Candida albicans, a suitable sequence to prepare DSO (dual specificity oligonucleotide) primers of this invention was determined. Forward and reverse primers were designed using the determined sequence. The symbol “I” denotes deoxyinosine in the following sequences.

CA-phr1-F1

(SEQ ID NO: 25)

TGCCGATGGTAGCAAAGGTG IIIII GGTGTTGCTT
CA-phr1-F2

(SEQ ID NO: 26)

TCCTCTGGTGGAAGCTCCAA IIIII GATCTTCCTC
CA-phr1-R1

(SEQ ID NO: 27)

CGTCCTATACAACAGAACCCTTCA IIIII GTTAGTCTTC

### Example I-8

### Primers for Amplifying Nucleic Acids of Haemophilus ducreyi

In the selected sequence of the p27 gene of Haemophilus ducreyi, a suitable sequence to prepare DSO (dual specificity oligonucleotide) primers of this invention was determined. Forward and reverse primers were designed using the determined sequence. The symbol “I” denotes deoxyinosine in the following sequences.

HD-p27-F1

(SEQ ID NO: 28)

TGCTTGCAACACCAAATGATG IIIII CAAAAAGTAGT
HD-p27-R1

(SEQ ID NO: 29)

TCTACAGGGTTGTTTGCAGGC IIIII ACAAGCAGTT
HD-p27-R2

(SEQ ID NO: 30)

GGTTTTGTTGCCATTCTTGGAA IIIII TGTAGTCTTC

### Example I-9

### Primers for Amplifying Nucleic Acids of Trichomonas vaginalis

In the selected sequence of the beta-tubulin 1 gene of Trichomonas vaginalis, a suitable sequence to prepare DSO (dual specificity oligonucleotide) primers of this invention was determined. Forward and reverse primers were designed using the determined sequence. The symbol “I” denotes deoxyinosine in the following sequences.

TV-btub1-F1

(SEQ ID NO: 31)

GCTGAATCCTGCGACTGCCT IIIII GCTTCCAGCT
TV-btub1-F2

(SEQ ID NO: 32)

CTCAACTCCGACCTTCGTAAGC IIIII GTCAACCTTG
TV-btub1-R1

(SEQ ID NO: 33)

GGAAGTGAGCGGATGTAAGGTAAG IIIII CGGCGTGGAT

### Example I-10

### Primers for Amplifying Nucleic Acids of Mycoplasma genitalium

In the selected sequence of the gyrA gene of Mycoplasma genitalium, a suitable sequence to prepare DSO (dual specificity oligonucleotide) primers of this invention was determined. Forward and reverse primers were designed using the determined sequence. The symbol “I” denotes deoxyinosine in the following sequences.

MG-gyrA-F1

(SEQ ID NO: 34)

ATAATCTTCAACATCGTGGTGGAG IIIII GTTAAAGGGC
MG-gyrA-R1

(SEQ ID NO: 35)

AATCTCATCATTTCCGTGGGTT IIIII TACTGAATACA
MG-gyrA-F2

(SEQ ID NO: 36)

AAAACCCACGGAAATGATGAGA IIIII ATTGGTTCTAC
MG-gyrA-R2

(SEQ ID NO: 37)

CTCCCTTAGCATTACGTTTTGTGA IIIII TATTTATCTATG

### Example I-11

### Primers for Amplifying Nucleic Acids of Treponema pallidum

In the selected sequence of the nucleotide sequence encoding 47-kd antigen of Treponema pallidum, a suitable sequence to prepare DSO (dual specificity oligonucleotide) primers of this invention was determined. Forward and reverse primers were designed using the determined sequence. The symbol “I” denotes deoxyinosine in the following sequences.

TP-47-F1

(SEQ ID NO: 38)

GCGTCATTTCAGGATTTGGG IIIII ACGGGGAGAT
TP-47-F2

(SEQ ID NO: 39)

TCACGGTATGAAGTTTGTCCCA IIIII GGTTCCTCAT
TP-47-R1

(SEQ ID NO: 40)

GCGTCATCACCGTAGTAGTCGTAG IIIII CGTGTTGAAG

### Example I-12

### Primers for Amplifying Nucleic Acids of Gardnella vaginalis

In the selected sequence of the internal transcribed spacer between 16S & 23S rRNAs of Gardnella vaginalis, a suitable sequence to prepare DSO (dual specificity oligonucleotide) primers of this invention was determined. Forward and reverse primers were designed using the determined sequence. The symbol “I” denotes deoxyinosine in the following sequences.

GV-F1

(SEQ ID NO: 41)

CGCTCGGTTGAGTGTGGTTAC IIIII TGGAAAACAA
GV-F2

(SEQ ID NO: 42)

TTGGCTTGTGTTCTTGGTGTTTG IIIII TTGAGAACTG
GV-R1

(SEQ ID NO: 43)

AATCCCACGACCCCGAATAC IIIII ACCTGACGGT

### Example I-13

### Preparation of Conventional Primers

With referring to the selected sequences, conventional primers were constructed to have no the DSO structure and then used as control primers. The names and sequences of conventional primers prepared are as follows:

TABLE 2

| Sequences of conventional primers | Names of |
|---|---|
| (5′ → 3′) | primers |
| TGC TCC AGC TAA AAG CGA AGG TGTTA | MH-gap-F1 |
| AAA CAG TTG TT | |
| ACT GTT TAG CTC CTA TTG CCA ACG | MH-gap-F2 |
| TATTG GAA AAA AAC TT | |
| GCC TGC TTT TGC ACC AAT AAT A TCACT | MH-gap-R2 |
| TGA TAC AAT TG | |
| AAA TTC CTC GTA AAG GCG GAC AAGGA | UU-F1 |
| ATG ATT AAA TCA | |
| GAA GCA CAC AAC AAA ATG GCG CATAC | UU-F2 |
| TGT GTA TTT CAC | |
| CAT AAC CCC CGC CCA TAC TAA TAGTT | UU-R1 |
| TGA AAA CAG GG | |
| GCT TTG GCT GAT GAT TGC GTA G TAATA | UU-R2 |
| ATG CAA CGT GC | |
| TAC GCC TGC TAC TTT CAC GCT GGAAA GTA | NG-porA-F1 |
| ATC AGA TG | |
| CAA TGG ATC GGT ATC ACT CGC TCTGC CGA | NG-porA-R1 |
| GCA AGA AC | |
| GACTCGGCTTGGGAAGAGCT TTTGC | Ctra-F279 |
| GGCGTCGTAT | |
| AGCCAGCACTCCAATTTCTGAC TGTGA | Ctra-R626 |
| GAATATATCA | |
| CCT TGG AGC ATT GTC TGG GC GATCA ACC | CT-F1 |
| AAT CCC G | |
| TGG ACC GCA TCA CTC AAC AA ACATA CTT | CT-R1 |
| GTA GAT C | |
| TGC AAC GGG TTA TTC ACT CCC AGTAA CAT | CT-F2 |
| TGA AAC TT | |
| ACC CAT ACC ACA CCG CTT TCT AAACC GCC | CT-R2 |
| TAC ACG T | |
| GCTTCTGGGAATACGACCTCTACT CTTTC | CT-ompA-F1 |
| AAAATTGGTAG | |
| CCAACACTCCAAGCAAAAGTA GTATC | CT-ompA-R1 |
| TGTATACAGT | |
| CCACATTCCCACAAAGCTGC ACGAG CTCCAACACT | CT-ompA-R2 |
| CAG CGG CTC ATC ATC GAA GA GCTGA CCC | HSV2-glyC-F1 |
| TGG AGA C | |
| TCT CCT GCG TCT GCG TGT GT ATCTG GGC | HSV2-glyC-R1 |
| CGG ATC G | |
| GCA TGG TCG CCC GTA AAC TC CATGG TGA | HSV2-glyC-R2 |
| TGG TTG G | |
| CAGCGGCTGATTATCGGCGA GGTGA CGCCCGCGAC | HSV1-gC-F1 |
| GCTCGTGCGTCTGCGTGTCG ATCTG GCCCGGGTTA | HSV1-gC-R1 |
| ACATGCCGGACCCCAAATTC CATGG TGATGGTTGG | HSV1-gC-R2 |
| TGCCGATGGTAGCAAAGGTG AATAT GGTGTTGCTT | CA-phr1-F1 |
| TCCTCTGGTGGAAGCTCCAA ATCTG GATCTTCCTC | CA-phr1-F2 |
| CGTCCTATACAACAGAACCCTTCA TATCG | CA-phr1-R1 |
| GTTAGTCTTC | |
| TGCTTGCAACACCAAATGATG AAGCA | HD-p27-F1 |
| CAAAAAGTAGT | |
| TCTACAGGGTTGTTTGCAGGC TTATC | HD-p27-R1 |
| ACAAGCAGTT | |
| GGTTTTGTTGCCATTCTTGGAA TTTTT | HD-p27-R2 |
| TGTAGTCTTC | |
| GCTGAATCCTGCGACTGCCT TCAGG GCTTCCAGCT | TV-btub1-F1 |
| CTCAACTCCGACCTTCGTAAGC TCGCT | TV-btub1-F2 |
| GTCAACCTTG | |
| GGAAGTGAGCGGATGTAAGGTAAG ACACA | TV-btub1-R1 |
| CGGCGTGGAT | |
| ATAATCTTCAACATCGTGGTGGAG TTGGG | MG-gyrA-F1 |
| GTTAAAGGGC | |
| AATCTCATCATTTCCGTGGGTT TTAAT | MG-gyrA-R1 |
| TACTGAATACA | |
| AAAACCCACGGAAATGATGAGA TTTTT | MG-gyrA-F2 |
| ATTGGTTCTAC | |
| CTCCCTTAGCATTACGTTTTGTGA GTCTA | MG-gyrA-R2 |
| TATTTATCTATG | |
| GCGTCATTTCAGGATTTGGG AGAGG ACGGGGAGAT | TP-47-F1 |
| TCACGGTATGAAGTTTGTCCCA GTTGC | TP-47-F2 |
| GGTTCCTCAT | |
| GCGTCATCACCGTAGTAGTCGTAG CGGAC | TP-47-R1 |
| CGTGTTGAAG | |
| CGCTCGGTTGAGTGTGGTTAC TGGTG | GV-F1 |
| TGGAAAACAA | |
| TTGGCTTGTGTTCTTGGTGTTTG GTGGT | GV-F2 |
| TTGAGAACTG | |
| AATCCCACGACCCCGAATAC GCAAC ACCTGACGGT | GV-R1 |

### Example II

### Preparation of Pathogen DNA Samples

Pathogens were prepared from affected swap or urine of patients and DNA was isolated from pathogens using the QIA quick PCR purification kit (Qiagen, USA).

### Example III

### Internal Control

The rbcL gene involved in photosynthesis of rice (Oryza sativa) was introduced into the pUC 18 vector and used as an internal control.

The sequences of primers for amplifying the rbcL gene were indicated in Table 3 and the amplified products were 719 bp in size.

TABLE 3

| | |
|---|---|
| Name | Sequence (5′- 3′) |

| | |
|---|---|
| rbcL-F164 | CTGCAGTAGCTGCCGAATCTTCIIIIIGTACATGGAC |
| rbcL-R882 | GTGAATGTGAAGAAGTAGGCCGTTIIIIIGGCAATAATG |

### Example IV

### Multiplex PCR

Using the primers prepared in Example I, the primer sets I and II were prepared as described below. Multiplex PCR amplifications were conducted using the primer sets I and II and the DNA samples of pathogens obtained from patients (Each primer set comprises primers for amplifying the internal control of Example III). As a negative control, multiplex PCR was performed by use of samples containing only the internal control.

TABLE 4
Primer Set I

| | | Size of PCR |
|---|---|---|
| Pathogens | Primer pair | products (bp) |
| Mycoplasma hominis | MH-gap-F2/MH-gap-R2 | 398 |
| Ureaplasma urealyticum | UU-F2/UU-R1 | 130 |
| Neisseria gonorrhoeae | NG-porA-F1/NG-porA-R1 | 214 |
| Chlamydia trachomatis | CTR-F279/CTR-R626 | 348 |
| HSV-2 | HSV2-glyC-F1/HSV2-glyC-R1 | 283 |

TABLE 5
Primer Set II

| | | Size of PCR |
|---|---|---|
| Pathogens | Primer pair | products (bp) |
| Candida albicans | CA-phr1-F2/CA-phr1-R1 | 234 |
| Haemophilus ducreyi | HD-p27-F1/HD-p27-R1 | 268 |
| Trichomonas vaginalis | TV-btub1-F1/TV-btub1-R1 | 580 |
| Mycoplasma genitalium | MG-gyrA-F1/MG-gyrA-R1 | 410 |
| Treponema pallidum | TP-47-F2/TP-47-R1 | 345 |
| Gardnerella vaginalis | GV-F2/GV-R1 | 509 |

The multiplex PCR amplifications were conducted using 20 μl of reaction mixtures containing the DNA sample, 2 μl of 10×PCR reaction buffer containing 15 mM MgCl₂ (Roche), 2 μl of dNTP (2 mM each dATP, dCTP, dGTP and dTTP), 4 μl of the primer set I or II and 0.5 μl of Taq polymerase (5 units/μl).

The tube containing the reaction mixture was placed in a preheated (94° C.) thermal cycler and the amplifications were then performed under the following thermal conditions: denaturation at 94° C. for 15 min followed by 30-45 cycles of 94° C. for 30 sec, 60-65° C. for 1.5 min and 72° C. for 1.5 min; followed by a 10-min final extension at 72° C.

PCR products were resolved by electrophoresis on an agarose gel containing EtBr and the bands formed were eluted for sequencing.

In addition, the multiplex PCR amplifications were carried out according to the same processes and conditions as described above, except that the conventional primer pairs (control primer set) prepared in Example I-13 were used instead of the primer set II.

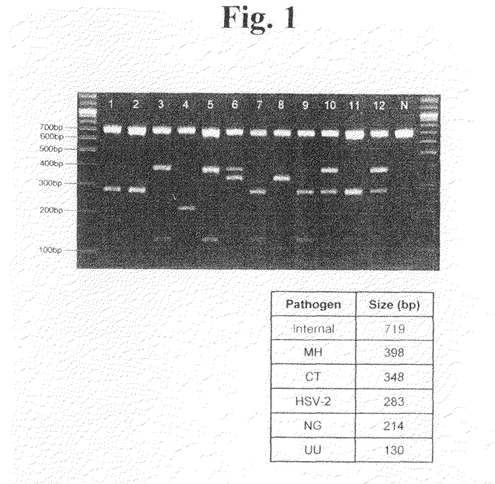

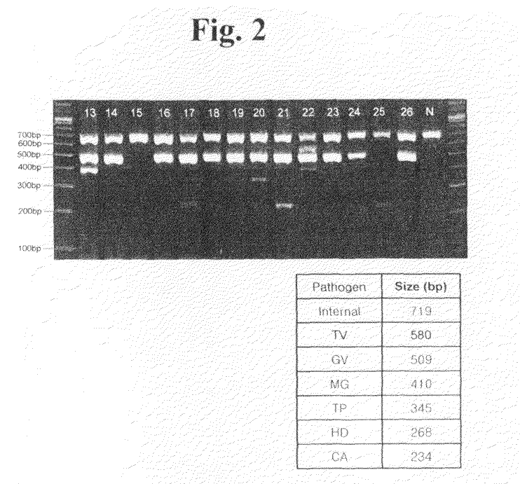

FIGS. 1 and 2 represent the results of multiplex PCR amplifications using the primer sets of this invention and pathogen samples from patients. As shown in FIGS. 1 (primer set I) and 2 (primer set II), the primer sets of this invention perfectly detect and identify the type of pathogens in the patients infected with sexually transmitted disease-causing pathogens without no-false positive results.

For detecting sexually transmitted disease-causing pathogens, the oligonucleotides of this invention are very useful as probes in microarray-based technologies as well as primers in PCR-based technologies.

The present invention provides oligonucleotides hybridizable with nucleic acids of pathogens causing sexually transmitted diseases, kits comprising them, and processes for amplifying and detecting pathogens using them. The oligonucleotides of this invention are designed to have the unique structure of the dual specific oligonucleotide (DSO). The oligonucleotides of this invention hybridize specifically with target nucleotide sequences of sexually transmitted disease-causing pathogens and are very useful in detecting sexually transmitted disease-causing pathogens according to multiplex PCR.

Having described a preferred embodiment of the present invention, it is to be understood that variants and modifications thereof falling within the spirit of the invention may become apparent to those skilled in this art, and the scope of this invention is to be determined by appended claims and their equivalents.

1. An oligonucleotide hybridizable specifically with a nuclei acid molecule of a sexually transmitted disease-causing pathogen, which is represented by the following general formula:
5′-Xₚ-Y_{q}-Zᵣ-3′
wherein, Xₚ represents a 5′-high Tₘ specificity portion having a hybridizing nucleotide sequence substantially complementary to a target sequence to hybridize therewith, Y_{q} represents a separation portion comprising at least two universal bases, Zᵣ represents a 3′-low Tₘ specificity portion having a hybridizing nucleotide sequence substantially complementary to a target sequence to hybridize therewith, p, q and r represent the number of nucleotides, and X, Y, and Z are deoxyribonucleotide or ribonucleotide; the Tₘ of the 5′-high Tₘ specificity portion is higher than that of the 3′-low Tₘ specificity portion, the separation portion has the lowest Tₘ in the three portions; the separation portion forms a non base-pairing bubble structure under conditions that the 5′-high Tₘ specificity portion and the 3′-low Tₘ specificity portion are annealed to the target sequence, enabling the 5′-high Tₘ specificity portion to separate from the 3′-low Tₘ specificity portion in terms of hybridization specificity to the target sequence, whereby the hybridization specificity of the oligonucleotide is determined dually by the 5′-high T_{M} specificity portion and the 3′-low Tₘ specificity portion such that the overall hybridization specificity of the oligonucleotide is enhanced; wherein the sexually transmitted disease-causing pathogen is Mycoplasma hominis, Ureaplasma urealyticum, Neisseria gonorrheae, Chlamydia trachomatis, Herpes simplex virus-1 or 2, Candida albicans, Haemophilus ducreyi, Trichomonas vaginalis, Mycoplasma genitalium, Treponema pallidum or Gardnella vaginalis; and wherein the target sequence is the nucleic acid molecule of the sexually transmitted disease-causing pathogen.

2. The oligonucleotide according to claim 1, wherein the universal base is selected from the group consisting of deoxyinosine, inosine, 7-deaza-2′-deoxyinosine, 2-aza-2′-deoxyinosine, 2′-OMe inosine, 2′-F inosine, deoxy 3-nitropyrrole, 3-nitropyrrole, 2′-OMe 3-nitropyrrole, 2′-F 3-nitropyrrole, 1-(2′-deoxy-beta-D-ribofuranosyl)-3-nitropyrrole, deoxy 5-nitroindole, 5-nitroindole, 2′-OMe 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2-′-O-methoxyethyl inosine, 2′O-methoxyethyl nebularine, 2′-O-methoxyethyl 5-nitroindole, 2′-O-methoxyethyl 4-nitro-benzimidazole, 2′-O-methoxyethyl 3-nitropyrrole, and combinations thereof.

3. The oligonucleotide according to claim 2, wherein the universal base is deoxyinosine, 1-(2′-deoxy-beta-D-ribofuranosyl)-3-nitropyrrole or 5-nitroindole.

4. The oligonucleotide according to claim 3, wherein the universal base is deoxyinosine.

5. The oligonucleotide according to claim 1, wherein the separation portion comprises contiguous nucleotides having universal bases.

6. The oligonucleotide according to claim 1, wherein the 5′-high Tₘ specificity portion is longer than the 3′-low Tₘ specificity portion.

7. The oligonucleotide according to claim 1, wherein the 5′-high Tₘ specificity portion is 15-40 nucleotides in length.

8. The oligonucleotide according to claim 1, wherein the 3′-low Tₘ specificity portion is 3-15 nucleotides in length.

9. The oligonucleotide according to claim 1, wherein the separation portion is 3-10 nucleotides in length.

10. The oligonucleotide according to claim 1, wherein the Tₘ of the 5′-high Tₘ specificity portion ranges from 40° C. to 80° C.

11. The oligonucleotide according to claim 1, wherein the Tₘ of the 3′-low Tₘ specificity portion ranges from 10° C. to 40° C.

12. The oligonucleotide according to claim 1, wherein the Tₘ of the separation portion ranges from 3° C. to 15° C.

13. The oligonucleotide according to claim 1, wherein the oligonucleotide comprises the nucleotide sequence selected from the group consisting of SEQ ID NOs:1-43.

14. An oligonucleotide pair hybridizable specifically with a nuclei acid molecule of a sexually transmitted disease-causing pathogen, wherein each oligonucleotide comprises each of the nucleotide sequences of SEQ ID NOs:1 and 3; each of the nucleotide sequences of SEQ ID NOs:2 and 3; each of the nucleotide sequences of SEQ ID NOs:4 and 6; each of the nucleotide sequences of SEQ ID NOs:4 and 7; each of the nucleotide sequences of SEQ ID NOs:5 and 6; each of the nucleotide sequences of SEQ ID NOs:5 and 7; each of the nucleotide sequences of SEQ ID NOs:8 and 9; each of the nucleotide sequences of SEQ ID NOs:10 and 11; each of the nucleotide sequences of SEQ ID NOs:12 and 13; each of the nucleotide sequences of SEQ ID NOs:14 and 15; each of the nucleotide sequences of SEQ ID NOs:16 and 17; each of the nucleotide sequences of SEQ ID NOs:16 and 18; each of the nucleotide sequences of SEQ ID NOs:19 and 20; each of the nucleotide sequences of SEQ ID NOs:19 and 21; each of the nucleotide sequences of SEQ ID NOs:22 and 23; each of the nucleotide sequences of SEQ ID NOs:22 and 24; each of the nucleotide sequences of SEQ ID NOs:25 and 27; each of the nucleotide sequences of SEQ ID NOs:26 and 27; each of the nucleotide sequences of SEQ ID NOs:28 and 29; each of the nucleotide sequences of SEQ ID NOs:28 and 30; each of the nucleotide sequences of SEQ ID NOs:31 and 33; each of the nucleotide sequences of SEQ ID NOs:32 and 33; each of the nucleotide sequences of SEQ ID NOs:34 and 35; each of the nucleotide sequences of SEQ ID NOs:36 and 37; each of the nucleotide sequences of SEQ ID NOs:38 and 40; each of the nucleotide sequences of SEQ ID NOs:39 and 40; each of the nucleotide sequences of SEQ ID NOs:41 and 43; or each of the nucleotide sequences of SEQ ID NOs:42 and 43.

15. A kit for detecting a sexually transmitted disease-causing pathogen, comprising the oligonucleotide of claim 1 or the oligonucleotide pair of claim 14.

16. A kit for amplifying a nucleic acid molecule of a sexually transmitted disease-causing pathogen, comprising the oligonucleotide of claim 1 or the oligonucleotide pair of claim 14.

17. A method for detecting a sexually transmitted disease-causing pathogen, which comprises hybridizing the oligonucleotide of claim 1 or the oligonucleotide pair of claim 14 with a nucleic acid molecule of a sexually transmitted disease-causing pathogen.
